(19) United States
(12) Patent Application Publication
(10) Pub. No.: US 2021/0072229 A1
(43) Pub. Date: Mar. 11, 2021

(54) NANOWIRE EVAULATION SYSTEMS AND METHODS FOR PREDICTING BEHAVIOR OF HYDROGELS AND MICROSYSTEM APPLICATION

(71) Applicants: Regents of the University of Minnesota, Minneapolis, MN (US); Technische Universität Dresden, Dresden (DE)

(72) Inventors: Bethanie Joyce Hills Stadler, Minneapolis, MN (US); Philipp Jan Mehner, Dresden (DE); Katinka Kohl, Dresden (DE); Andreas Richter, Dresden (DE)

(73) Assignees: Regents of the University of Minnesota, Minneapolis, MN (US); Technische Universität Dresden, Dresden (DE)

(21) Appl. No.: 17/017,802

(22) Filed: Sep. 11, 2020

Related U.S. Application Data
(60) Provisional application No. 62/898,647, filed on Sep. 11, 2019.

(51) Int. Cl. G01N 33/50; B01L 3/00; C12M 3/06

(52) U.S. Cl. CPC G01N 33/5044; C12M 23/16; B01L 3/502715

(57) ABSTRACT

Systems and methods for evaluating one or more characteristics or parameters of a material, such as a soft material (e.g., hydrogel, human cell, UV-curable polymer, etc.). Methods include incorporating a plurality of magnetic nanowires into the material to form a test solution. The test solution is subjected to a magnetic field. A change in the magnetic nanowires in response to the magnetic field is recorded. A characteristic of the material is determined based upon the recorded change. In some embodiments, the applied magnetic field causes the magnetic nanowires to rotate from an initial orientation to a stimulated orientation, with the change in orientation being indicative of a stiffness (e.g., internal stiffness) of the material.

### RELATED APPLICATIONS

This Non-Provisional patent application claims the benefit of the filing date of U.S. Provisional Patent Application Ser. No. 62/898,647, filed Sep. 11, 2019, entitled “HYDROGEL-BASED MICRO-VALVES AND METHODS OF USE, SYSTEMS AND METHODS FOR PREDICTING BEHAVIOR THEREOF, AND MICROSYSTEM APPLICATIONS,” the entire teachings of which are incorporated herein by reference.

### BACKGROUND

The difficulty in advancing lab on chip applications is the lack of reliable miniaturized fluidic control components. Normally, microfluidic systems consist of passive components, like inlets, outlets, channels and reaction chambers. Active components like pumps and valves are mostly externally connected, preventing an upscaling of those passive microfluidic systems. Therefore, hydrogel-based micro-valves enable an intriguing solution to provide a chemical triggered transistor for microfluidics analogous to an electronic transistor as known in the computer industry. First fluidic logic gates have developed recently and are described, for example by P. Frank et al., “Autonomous Integrated Microfluidic Circuits for Chip-Level Flow Control Utilizing Chemofluidic Transistors,” Adv. Funct. Mater., vol. 27, no. 30, p. 1700430, (Aug. 2017); P. N. Duncan et al., “Scaling of pneumatic digital logic circuits,” Lab Chip, vol. 15, no. 5, pp. 1360-1365, (2015), the entire teachings of which are incorporated herein by reference. These investigations revealed that further chemo-fluidic logic development is challenging.

### SUMMARY

The inventors of the present disclosure recognized that a need exists for addressing the problems associated with characterizing parameters, such as stiffness, of certain material, such as soft materials. In some examples, methods of the present disclosure include incorporating a plurality of magnetic nanowires into the material to form a test solution. The test solution is subjected to a magnetic field. A change in the magnetic nanowires in response to the magnetic field is recorded. A characteristic of the material is determined based upon the recorded change. In some embodiments, the applied magnetic field causes the magnetic nanowires to rotate from an initial orientation to a stimulated orientation, with the change in orientation being indicative of a stiffness (e.g., internal stiffness) of the material.

### BRIEF DESCRIPTION OF THE DRAWINGS

FIG. 1 schematically illustrates a measurement system or setup in accordance with principles of the present disclosure;

FIG. 2 schematically illustrates another measurement system in accordance with the principles of the present disclosure;

FIG. 3 is a plot of light intensity over time for a hydrogel material subjected to the methods of the present disclosure;

FIG. 4 schematically illustrates another measurement system in accordance with the principles of the present disclosure;

FIG. 5A is a TEM image of a multilayer nanowire;

FIG. 5B is an SEM image of a nanobots as grown;

FIG. 5C is an image of osteosarcoma-internalized nanobots;

FIG. 6A are still shots from a video taken during performance of nanobot evaluation methods of the present disclosure and showing scattering of polarize red light off nanobots;

FIG. 6B is a plot of brightness of the reflected beam vs. time during the method giving rise to the still shots of FIG. 6A;

FIG. 6C is a plot of shear modulus of the polymer utilized in the method giving rise to the still shots of FIG. 6A vs. exposure time.

### DETAILED DESCRIPTION

Some features of the present disclosure provide systems and methods for measuring one or more parameters or characteristics, for example stiffness, of hydrogels and other soft materials. As a point of reference, inertial effects of fluid and transport effects of the stimuli can play a major role in designing microfluidic systems. If the swelling of a hydrogel is used to perform a transistor-like behavior, then its chemistry defines a nonlinear swelling behavior that needs to be predictable to design a targeted chip application. Due to this complexity, some aspects of the present disclosure relate to or provide a simplified and efficient model based on finite element modelling and experimental data which are further progressed to a reduced order model for efficient computation. In some embodiments, finite element modelling of the present disclosure can be akin to explanations provided by P. J. Mehner et al., “Finite Element Model of a Hydrogel-Based Micro-Valve,” Volume 2: Modeling, Simulation and Control; Bio-Inspired Smart Materials and Systems; Energy Harvesting, (2016), the entire teachings of which are incorporated herein by reference. In some embodiments, reduced order models of the present disclosure can be akin to explanations provided by P. J. Mehner et al., “Reduced order model of a hydrogel-based microvalve with pressure, flow and concentration correlations,” 2018 Symposium on Design, Test, Integration & Packaging of MEMS and MOMS (DTIP), pp. 1-5 (2018), the entire teachings of which are incorporated herein by reference.

With some hydrogel-based micro-valve models, for example as implicated above, calculation of the resistance is targeted to be redefined considering the current geometry, energy loss due to recirculation zones (for example), and leakage due to high pressure. However, a description of the stiffness of the hydrogel material is still missing. Measuring or evaluating stiffness of a hydrogel material or the like can be beneficial for many other end-use applications. By way further background, multiple methods exits to measure the stiffness of hydrogels. In macroscopic systems, the elastic properties are usually determined by tensile and shear testing experiments on larger specimens. In microscopic systems, not only the specimens but also the test equipment, in particular the probe tips, must be miniaturized. Clamping of samples to measure the force-displacement relationship is often difficult or even impossible, especially for small and highly flexible materials such as hydrogels. Alternatively, the stiffness of miniaturized samples has been detected through micro-indentation testing, or based on the atomic force microscopy. The atomic force microscope (AFM) is utilized to detect the local surface stiffness of the material through colloidal probing of small gels with micrometer size. In summary, both micro-indentation and atomic force-based testing methods utilize tiny tips or needles to measure the force-displacement relationship at the surface of the specimens.

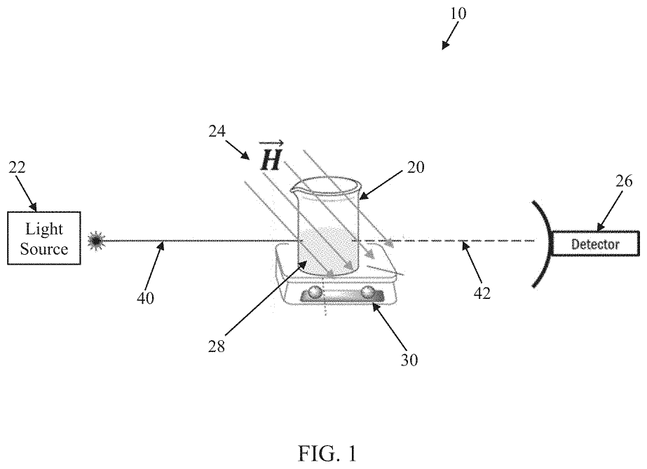

Against the above background, some systems and methods of the present disclosure determine stiffness parameters in the interior of a hydrogel or other material using contactless measuring techniques. For example, some systems and methods of the present disclosure employ magnetic nanowires (also referred to herein as “nanobots”), and applying an alternating field and monitoring the frequency of vibrating nanowires. The magnetic nanowires can have various forms and/or constructions, such as single striped or multilayered nanowire of ferromagnet/non-magnet layers (e.g., gold-tipped nickel nanowires or nanobots). In some non-limiting embodiments, the magnetic nanowires can have a length on the order of 22-35 um, and a diameter on the order of 40-200 nm. One example of a measurement system 10 in accordance with principles of the present disclosure is shown in FIG. 1. The system 10 includes a sample reservoir 20, a light source 22, a magnetic field device (referenced generally at 24), and a detector 26. The sample (e.g., hydrogel) to be measured or evaluated is held in the reservoir 20, along with a multiplicity of the magnetic nanowires as described above. The so-formulated test solution is identified at 28 in FIG. 1. The reservoir 20 is formed of a material selected to be transparent or substantially transparent (i.e., within 10% of truly transparent) to light from the light source 22. The magnetic nanowires can be mixed into the sample in various manners, such as via a stirring plate 30. The light source 22 can assume various forms, and in some embodiments is a laser light source or other device capable of emitting coherent light, such as a commercial laser (e.g., a commercial red laser diode) with constant intensity. The magnetic field device 24 can assume a wide variety of forms known in the art and capable of establishing an alternating magnetic field (\vec{H}) through the sample as contained within the reservoir 20. The detector 26 can be an available camera or the like (e.g., a digital camera) capable of capturing expected light fluctuation. The light source 22 is arranged to emit light 40 to the reservoir 20, interacting with the sample/magnetic nanowire solution 28 contained therein. In some embodiments, one or more optical elements can be located optically between the light source 22 and the reservoir 20 as described below. Regardless, methods of the present disclosure can include the nanowires incorporated in to the sample initially being aligned, and through the magnetic field (\vec{H}) are rotated. The resultant angle rotation is visualized through reflection of the light 42 as received at the detector 26 (e.g., depending on the orientation of the detector 26, changes in either reflected or transmitted light can be observed). The measured angle rotation of the magnetic nanowires is replicated (e.g., a numerical model in ANSYS Mechanical (an FEM Software tool), and the Young's Modulus of the sample can be determined by matching the measure angle and the simulated angle of the magnetic nanowires. With this method the viscoelastic behavior can be observed, and the results can later be implemented in the reduced order model to predict maximum closing pressures and maximum operating pressures of a high number of active elements.

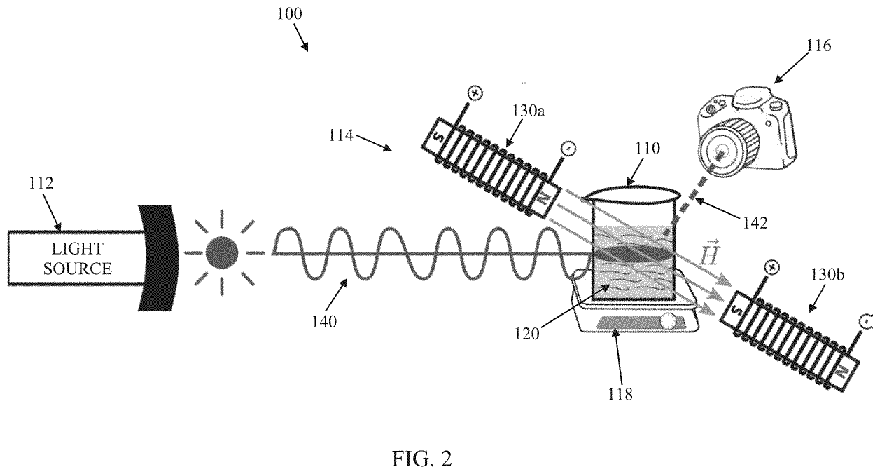

Another embodiment of a measurement system 100 in accordance with principles of the present disclosure is shown in FIG. 2. The system 100 includes a reservoir 110, a light source 112, a magnetic field device 114 (referenced generally), and a detector 116. The sample (e.g., hydrogel) to be measured or evaluated is held in the reservoir 110, along with a multiplicity of the magnetic nanowires as described above. The reservoir 110 is formed of a material selected to be transparent or substantially transparent (i.e., within 10% of truly transparent) to light from the light source 112. The magnetic nanowires can be mixed into the sample in various manners, such as via a stirring plate 118 (e.g., a stirring plate capable of operating at rotational rate on the order of 60-1200 rpm). The resulting test solution is identified at 120 in FIG. 2.

The light source 112 can assume various forms, and in some embodiments is a laser light source or other device capable of emitting coherent light, such as a commercial laser (e.g., a helium-neon laser device operating at a wavelength on the order of 632.8 nm and a power output (Pₒᵤₜ) on the order of 4.5 mW in some examples). In some embodiments, one or more optical elements can be located optically between the light source 112 and the reservoir 110 as described below.

The magnetic field device 114 can assume a wide variety of forms known in the art and capable of establishing an alternating magnetic field (\vec{H}) through the sample as contained within the reservoir 110. For example, the magnetic field device 114 can include two or more electromagnets 130a, 130b with polarities arranged as shown relative to the reservoir 110 (and thus the sample/magnetic nanowires). The electromagnets 130a, 130b are operated by a controller (not shown) to create the magnetic field (\vec{H}).

The detector 116 can be an available camera or the like capable of capturing expected light fluctuation. In some non-limiting examples, the detector 116 can be a digital camera with a frame rate (frames per second) or frequency of at least 240 Hz.

With some methods of the present disclosure, the system 100 is operated to initially arrange all of the magnetic nanowires in the test solution 120 (as contained in the reservoir 110) to have the same orientation. This alignment can be achieved by a strong and homogenous magnetic field impulse applied through a vibrating sample magnetometer (VSM). The electromagnets 130a, 130b are arranged to be perpendicular to the so-aligned nanowires; the applied magnetic field (\vec{H}) creates a torque, depending upon the applied current signal. When the electromagnets 130a, 130b (or other magnetic field device) are activated, the magnetic nanowires tile slightly toward the poles. After switching off the magnetic field off, the magnetic nanowires returning nearly to the starting positon can be indicative of the sample material (in which the magnetic nanowires are embedded) behaves almost elastic. The tilt angle of the magnetic nanowires in the presence of the magnetic field depends on the applied torque, the size of the nanowires, and on the stiffness of the sample material. With this in mind, the light source 112 is operated to emit light 140 into the test solution 120 (as contained in the reservoir 110), and light 142 as reflected off of the solution 120 or transmitted through the solution 120 is detected by the detector 116. An angle of inclination of the nanowires can be determined from the so-collected light 142. In some embodiments, the light reflected can be based on the magneto-optic Kerr-Effect. The stimulation process can be repeated several times by a pulse function to observe results over many cycles. In other embodiments, depending upon the orientation of the detector 116, changes in light transmitted through the test solution 120 is detected or collected at the detector 116 and utilized to determine the angle of inclination.

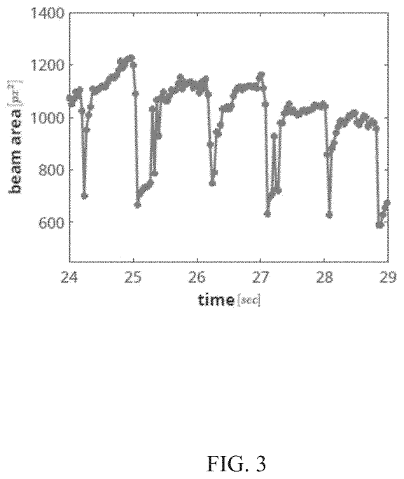

The light intensity measured by the detector 116 is inversely proportional to the deflection amplitudes of the magnetic nanowires. A typical transient response generated by some systems and methods of the present disclosure in measuring properties of a hydrogel material sample is shown in FIG. 3. The pulse frequency of the current applied to the electro magnets 130a, 130b was about 1 Hz and the high value was about 10% of the pulse duration. The light intensity was observed as decreasing with increasing tilt angles (which depends on the optical set up). Finally, the signal difference between high and low value is proportional to the tilt amplitude. As a point of reference, a small signal drift can be seen in the results of FIG. 3, which is superimposed to the pulse signal response. The light intensity increases over time, which means that the angle of inclination slowly increased during the measurements. The behavior reverses when the current at the electromagnets 130a, 130b changes the direction, indicating a viscoelastic behavior of the hydrogel sample material. Regardless, the measurement of FIG. 3 represents how far the nanowires can deflect within a material sample, such as a hydrogel. The rotation angle can be replicated in a finite element model where this angle of inclination to the applied magnetic force is adapted by changing the ambient material stiffness. In some embodiments, the controller of the magnetic field device 114 is programmed or operated to effect a magnetic field sufficient for partial (i.e., less than full) rotation or turning of the magnetic nanowires; since full turns of the magnetic nanowires in solid material may be unreasonable for stiffness measurements, the nanowires are, in some embodiments, stimulated to small tile angles or vibration amplitudes.

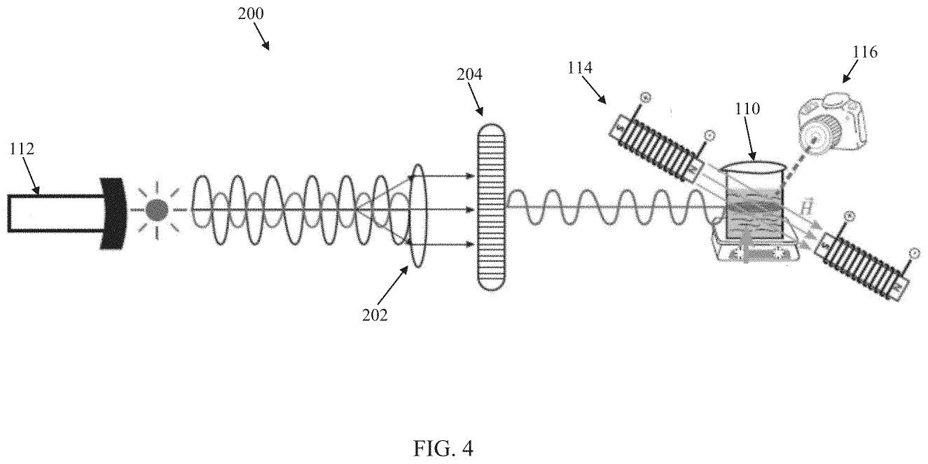

Another embodiment of a measurement system 200 in accordance with principles of the present disclosure is shown in FIG. 4. The system 200 can be akin to the system 100 (FIG. 2), described above, and includes the reservoir 110, the light source 112, the magnetic field device 114 (referenced generally), and the detector 116 that are configured and operate in accord with the descriptions above. In addition, the system 200 includes various optical elements that effect light from the light source 112 in a desired manner for interfacing with the magnetic nanowire-laden sample (as contained in the reservoir 110). For example, the system 200 can include one or more condenser lenses 202 (e.g., +45° condenser lense available from Thorlabs under the trade designation LPNIR050-MP) and a waveguide 204 (e.g., a half-wave plate available from Newport under the trade designation PR5OPP). Other optical elements can also be employed as will be understood by one of ordinary skill.

Contrary to common rheology testing, some systems and methods of the present disclosure provide for contactless measurement. The magnetic nanowires with their rotation as described above only apply minimal force to the sample under inspection (e.g., a hydrogel) to determine the core stiffness of the material. A classical rheology measurement system consists of a cone-plate arrangement, and through the momentum needed to create an oscillating movement, the shear relaxation modulus, the shear storage modulus, and the shear loss modulus of the material are determined. During the rheology experiment, the applied pressure to the material affects the characterization results.

The systems and methods of the present disclosure can be useful with numerous end-use applications. For example, being able to model and predict the behavior of complex polymeric micro-fluidic systems via evaluation of hydrogels under consideration can help to reduce the experimental effort and decrease the development time of microfluidic systems by material libraries and computer aided design process. Other end use applications for evaluating “soft” or very soft materials have been devised as described below.

Some embodiments of the present disclosure relate to applications of micro-systems, for example probing internal cancer cell mechanics via magnetic nanobots. As a point of reference, cancer is the leading cause of death worldwide, accounting for approximately 13% of all deaths, and the number of cases are expected to double by 2030. Researchers have been working on a cure for decades, but the goal of a total cure appears to be far off. However, major advances have been made in understanding cell transformations, diagnosing tumors, and developing successful therapies. Some aspects of the present disclosure provide innovative nanobots that will probe cells from the inside to diagnosis and treat cancer.

Heterogeneity has recently emerged as an important paradigm in cancer research. Different types of cancers are often united by common themes, also characterized by inherent differences in epidemiological factors, molecular mechanisms, and clinical features. In other words, it can be important to understand the physical features of each tumor type and the cancer cells of which they are composed to seek diagnosis and therapy. The National Cancer Institute has posed this provocative question: “How can the physical properties of tumors, such as a cell's electrical, optical or mechanical properties, be used to provide earlier or more reliable cancer detection, diagnosis, prognosis, or monitoring of drug response or tumor recurrence?”.

Currently, mechanical measurements of cells use external probes, such as atomically sharp atomic force microscope (AFM) tips. These are important pioneering studies, but they require that the cell is attached to a substrate and/or probes. The bonding itself has been found to change the cell modulus, or viscosity, by an order of magnitude. So, how can one measure the properties of a cell while it is floating in solution, or even while it is inside tissue? In some aspects of the present disclosure, the use of cell-internalized nanobots to characterize heterogeneous cancer cell types are provided.

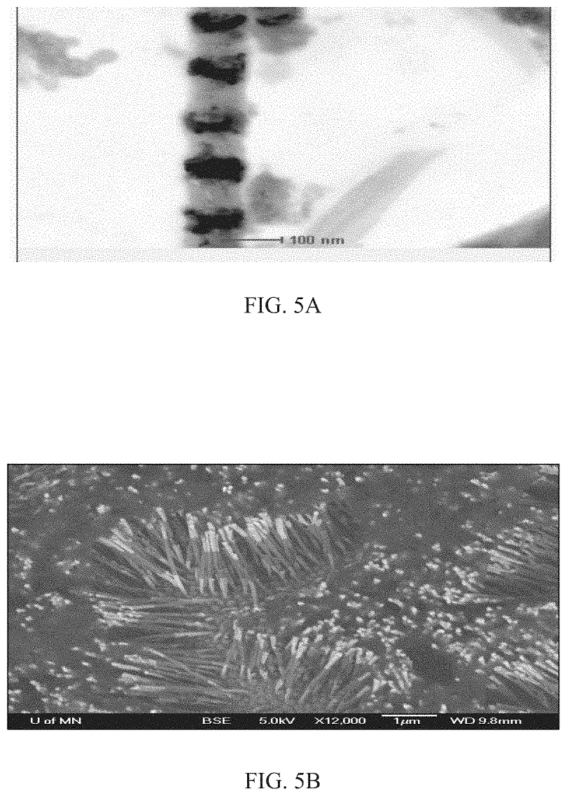

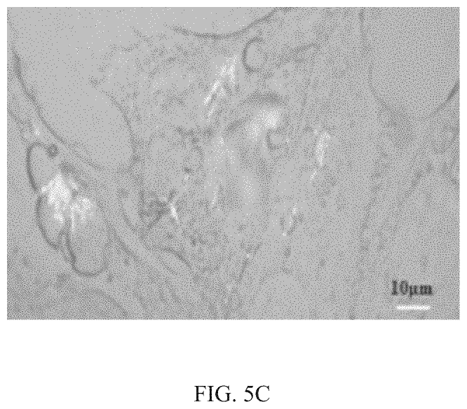

In some embodiments, billions of nanobots are synthesized by electrodeposition inside templates, after which the templates are dissolved away as reflected by FIGS. 5A-5C. FIG. 5A depicts a single striped (or multilayered) nanowire of ferromagnet/nonmagnet layers as observed by TEM. FIG. 5B illustrates an SEM image of gold-tipped nickel nanobots as grown, immediately after dissolving the template to expose the nanobots along with schematic representations to assist the reader visualize the nanobots. FIG. 5C depicts osteosarcoma-internalized nanobots. Once dispersed in culture, these nanobots are internalized by cells, where they can be manipulated by external magnetic fields to act as nanobot probes as described above. As a point of reference, the design and synthesizing of nanowires for magnetoelectronics has been considered for some. Non-limiting examples are provided by M. Hein et al., “Fabrication of Biolnspired Inorganic Nanocilia Sensors,” IEEE Transactions on Magnetics 49 191-4 (2013); A. Sharma et al. “Magnetic Barcode Nanowires for Osteosarcoma Cell Control, Detection, and Separation,” IEEE Transactions on Magnetics 49 453-6 (2013); M. Maqableh et al., “Low Resistivity 10 nm Diameter Magnetic Sensors,” Nano Letters 12 4102-4109 (2012); M. Maqablah et al., “CPP GMR through Nanowires (Invited),” IEEE Transactions on Magnetics 48 1-7 (2012); K. Sai Madhukar Reddy et al., “Electrochemical Synthesis of Magnetostrictive Fe—Ga/Cu Multilayered Nanowire Arrays with Tailored Magnetic Response,” Advanced Functional Materials 21 4677 (2011); J. Zou et al., “Nanoporous Silicon with Long-Range-Order using Imprinted Anodic Alumina Etch Masks,” Applied Physics Letters 89, 093106 (2006); P. D. McGary et al., “Magnetic Nanowires for Acoustic Sensors (Invited)” Journal of Applied Physics 99, 08B310 (2006); the entire teachings of each of which are incorporated herein by reference.

A wide range of heterogeneous cancer cells have been catalogued by aggressiveness. These cells include canine and human osteosarcoma (bone cancer) and hemangiosarcoma (endothelial—from skin, spleen, heart, lung, liver, and brain), among others. Some examples are provided, for example, by J. F. Modiano et al, “Predictive value of p16 or Rb inactivation in a model of naturally occurring canine non-Hodgkin's lymphoma,” Leukemia 21, 184-187 (2007); S. P. Fosmire et al., “Interaction of the p16 cyclin-dependent kinase inhibitor in high-grade canine non-Hodgkin's T-cell lymphoma” Vet Pathol 44, 467-478 (2007); B. H. Gorden et al., “Identification of three molecular and functional subtypes in canine hemangiosarcoma through gene expression profiling and progenitor cell characterization,” Am J Pathol in press (2014); B. A. Tamburini et al., “Gene expression profiling identifies inflammation and angiogenesis as distinguishing features of canine hemangiosarcoma” BMC Cancer 10, 619 (2010); A. L. Sarver et al., “MicroRNAs at the human 14q32 locus have prognostic significance in osteosarcoma,” Orphanet journal of rare diseases 8, 7 (2013); M. C. Scott et al., “Molecular subtypes of osteosarcoma identified by reducing tumor heterogeneity through an interspecies comparative approach,” Bone 49, 356-367 (2011); V. Thayanithy et al., “Combinatorial treatment of DNA and chromatin-modifying drugs cause cell death in human and canine osteosarcoma cell lines,” PLoS ONE 7, e43720 (2012); the entire teachings of each of which are incorporated herein by reference.

Some aspects of the present disclosure include or provide for the manipulation of the nanobots using external fields in order to probe mechanical properties. Nanomechanical measurements may also find use in block copolymers and other future applications.

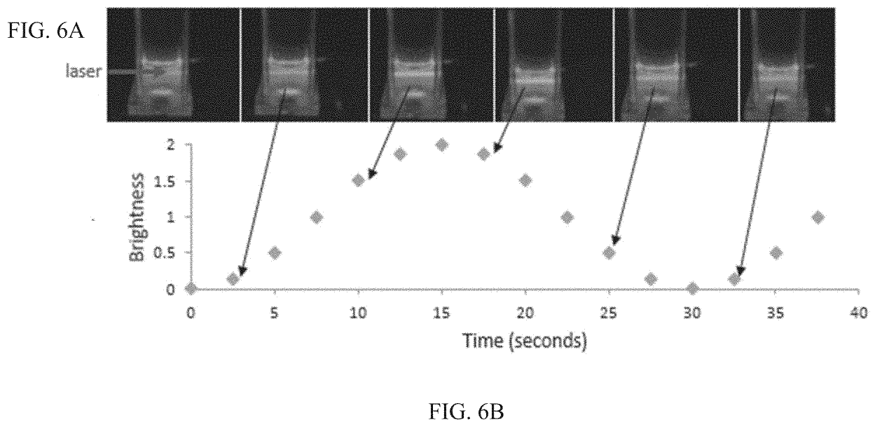

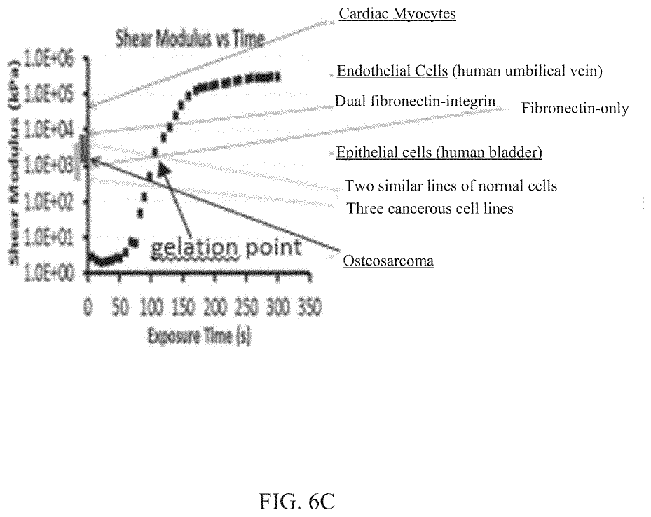

Some aspects of the present disclosure relate to or provide for the manipulation (e.g., rotation) of nanobots as described above to measure mechanical properties. In some embodiments, magnetic nanobots in a UV-curing polymer are manipulated using a rotating magnetic field as the polymer cures, and measurements are made (e.g., a red laser beam captures the motion of the nanowires as a double frequency “blinking”). For example, FIG. 6A provides still shots of a movie showing scattering of polarized red light off nanobots in a UV curing polymer as the suspension was subjected to both a UV light source and a 1 rpm rotating magnetic field. FIG. 6B is a plot of the brightness of the reflected beam vs time showing the double frequency “blinking” expected when the nanobots were rotating. It was observed that after gelation, the nanobots merely rocked back-and-forth in response to the field, and once the polymer turned glassy the nanobots were immobile. FIG. 6C is a plot of shear modulus of the UV curing polymer vs. exposure time as it changed from resin to gel to glass as compared to moduli of several cells.

Nanobots can be synthesized and magnetically characterized, for example as described above. These nanobots can then be incubated with malignant cells in a sterile environment. For example, Gold-Ni nanobots with diameters of 13, 50, and 100 nm and lengths of 0.1, 1, 5, and 10 um can be used to vary the shape-dependent viscous drag inside the cytoplasm as a magnetic field is rotated with increasing frequency. Nanobots will start and stop spinning at characteristic frequencies depending on both their shape and the modulus of the cytoplasm for each cell. The motion of nanobots in various UV-curing polymers can be calibrated. A library of the motion experienced by nanobots with various moments, lengths, diameters in viscoelastic media vs external fields of varying strength and rotation rates can be prepared. Theoretical calculations can be compared to experimental data as the cell cytoplasms are characterized.

The measurements as described above can yield a transformative understanding of cancer mechanics to characterize the heterogeneity of cancer. Literature reports that healthy cells are mechanically stiffer than cancer cells, and therefore healthy cells can resist internal nanobot rotations better than malignant cells. Therefore, it is believed that using properly designed nanobots and rotating magnetic fields, but not requiring specific cancer biomarkers, some features of the present disclosure provide for the ability to kill cancer cells without killing healthy cells. Functionalized nanobots can also target cells, but it is hypothesize that it may not be necessary and accidental uptake by other cells may not be harmful.

Regardless of the soft material(s) being measured or evaluated (e.g., hydrogels, human cells, UV-curing polymers, etc.), the systems and methods of the present disclosure provide a marked improvement over previous designs. Incorporating magnetic nanowires (or nanobots) into the soft material in question and evaluating partial rotation in the presence of a magnetic field facilitates an understanding of the material's stiffness parameters or characteristics in ways not previously available. Conventional measurement techniques measure surface stiffness (no core stiffness or average stiffness) through indentation on the surface, requiring special tips or other highly expensive devices. Rheometry techniques would destroy many types of soft materials. Tensile testing is not viable with many soft material because clamping soft materials is difficult at best, and millimeter scaled sample measurements are next to impossible. The systems and methods of the present disclosure overcome these, and many other, concerns.

Although the present disclosure has been described with reference to preferred embodiments, workers skilled in the art will recognize that changes can be made in form and detail without departing from the spirit and scope of the present disclosure.

What is claimed is:

1. A method of evaluating a characteristic of a material, the method comprising:
incorporating a plurality of magnetic nanowires into the material to form a test solution;
subjecting the test solution to a magnetic field;
recording a change in the magnetic nanowires in response to the magnetic field;
determining a characteristic of the material based upon the recorded change.

2. The method of claim 1, wherein following the step of incorporating a plurality of magnetic nanowires into the material and prior to the step of subjecting the test solution to a magnetic field, the method further comprising:
aligning the magnetic nanowires within the test solution.

3. The method of claim 2, wherein the step of aligning includes applying a homogenous magnetic field impulse to the test solution.

4. The method of claim 3, wherein the step of applying a homogenous magnetic field impulse includes operating a vibrating-sample magnetometer.

5. The method of claim 2, wherein the step of aligning the magnetic nanowires include causing the magnetic nanowires to assume an initial orientation, and the step of subjecting the test solution to a magnetic field includes causing the magnetic nanowires to rotate from the initial orientation to a stimulated orientation.

6. The method of claim 5, further comprising determining a difference between the initial orientation and the stimulated orientation.

7. The method of claim 6, wherein the step of determining a characteristic is based upon the determined difference between the initial orientation and the stimulated orientation.

8. The method of claim 7, further comprising sequentially repeating the steps of aligning the nanowires to the initial orientation, causing the nanowires to rotate from the initial orientation to a stimulated orientation, and determining a difference between the initial orientation and the stimulated orientation.

9. The method of claim 7, wherein the determined difference between the initial orientation and the stimulated orientation is a measured angle of rotation, and further wherein the step of determining a characteristic includes matching the measured angle of rotation with a simulated angle of rotation.

10. The method of claim 1, wherein the step of recording a change in the magnetic nanowires in response to the magnetic field includes:
emitting light into the test solution; and
detecting light from the test solution.

11. The method of claim 10, wherein the step of emitting light includes operating a light source to emit a laser beam into the test solution.

12. The method of claim 10, wherein the light from the test solution is one of light reflected by the test solution and light transmitted through the test solution.

13. The method of claim 1, wherein the characteristic is a stiffness of the material.

14. The method of claim 1, wherein the material is a hydrogel.

15. The method of claim 14, wherein the characteristic is an interior stiffness of the hydrogel.

16. The method of claim 1, wherein the material includes a human cell.

17. The method of claim 1, wherein the material is a UV-curable polymer.

18. A method of evaluating a microfluidic system including a microvalve formed of a hydrogel, the method comprising:
determining a characteristic of the hydrogel according to the method of claim 1; and
characterizing a behavior of the microvalve based upon the determined characteristic.

19. The method of claim 18, wherein the behavior includes at least one parameter selected from the group consisting of maximum closing pressure and maximum operating pressure.

20. The method of claim 18, further comprising predicting performance of the microfluidic system based upon the characterized behavior of the microvalve.
